(19) United States
(12) Patent Application Publication
(10) Pub. No.: US 2001/0023319 A1
(43) Pub. Date: Sep. 20, 2001

(54) Guide wire used for medical treatment

(75) Inventors: Naohiko Miyata, Nagoya-shi (JP); Masashi Momota, Kamakura-shi (JP); Satoshi Nagano, Osaka (JP)

Correspondence Address: Donald S. Dowden, Cooper & Dunham LLP, 1185 Avenue of the Americas, New York, NY, 10036, US

(73) Assignee: ASAHI INTECC CO., LTD.

(21) Appl. No.: 09/862,934

(22) Filed: May 22, 2001

Related U.S. Application Data
(63) Continuation-in-part of application No. 08/881,643, filed on Jun. 24, 1997.

(51) Int. Cl. A61B 8/14

(52) U.S. Cl. 600/462; 600/459

(57) ABSTRACT

A guide wire for medical treatment has a flexible main wire and a coil spring in which the main wire is inserted. The coil spring has an untransmissive part and a transmissive part and is manufactured by welding together under a proper welding load a wire made of a material through which radiation cannot pass and a wire made of a material through which radiation can pass to form a base wire, which is coiled to form the coil spring. The guide wire can be smoothly curved and smoothly inserted or extracted into or from the blood vessel without injuring the blood vessel.

### CROSS REFERENCE TO A RELATED APPLICATION

This is a continuation-in-part of application Ser. No. 08/881,643, filed Jun. 24, 1997. Applicants claim the benefit of that application under 35 U.S.C. §120.

### FIELD OF THE INVENTION

The present invention relates to a guide wire used for medical treatment to introduce a catheter into a blood vessel in the heart.

More particularly, the present invention relates to a guide wire used for medical treatment consisting of a flexible main wire and a coil spring in which the top of the main wire is inserted. The coil spring consists of an untransmissive coil part and a transmissive coil part and is manufactured by a process comprising connecting an untransmissive wire made of a material through which radiation cannot pass and a transmissive wire made of a material through which radiation can pass. The connection is made by welding the wires together to form a base wire, and the base wire is coiled to form the coil spring.

### DESCRIPTION OF THE PRIOR ART

A guide wire consisting of a flexible wire has been used to insert safely and certainly a catheter consisting of a flexible very thin tube into a blood vessel.

A conventional guide wire 1 shown in FIG. 5 consists of a main wire 2 and a coil spring 3 in which the top of the main wire is inserted, wherein the coil spring 3 consists of an untransmissive coil part 4 made of an untransmissive wire and a transmissive coil part 5 made of a transmissive wire, and the untransmissive wire and the transmissive wire are connected together by screwing in or brazing.

In the conventional guide wire 1, since the untransmissive coil part 4 and the transmissive coil part 5 are connected together by screwing in or brazing, very fine and troublesome work is necessary to connect a pair of very small coils together. In the case of a screwing connection, an overlap of the untransmissive part 4 and the transmissive part 5 is formed in a connecting part 6. In the case of brazing connection, the filler spreads and attaches to the untransmissive part 4 and the transmissive part 5 in the connecting part 6, the part around the connecting part 6 is hardened by the heat of brazing, and the overlap part and the part to which the filler attaches and is heated by brazing become rigid parts 14 as shown in FIG. 6a.

Accordingly, when the guide wire 1 is inserted into the blood vessel 15 to curve along the blood vessel 15 as shown in FIG. 6b, an angular part is formed around the rigid part 14 to injure the blood vessel 15, and it becomes difficult to insert and extract said guide wire 1 into/from the blood vessel.

### SUMMARY OF THE INVENTION

Accordingly, an object of the present invention is to save the very fine and troublesome work to connect the untransmissive coil part and the transmissive coil part together to produce the coil spring of the guide wire.

A further object of the invention is to provide a coil spring that gives the guide wire a smooth curve without an angular part to insert or extract the guide wire smoothly into or from the blood vessel without injuring the blood vessel.

Briefly, the objects of the present invention can be attained by a guide wire used for medical treatment comprising a flexible main wire and a coil spring in which the top of the main wire is inserted wherein the coil spring consists of an untransmissive coil part and a transmissive coil part and is manufactured by a process comprising connecting an untransmissive wire made of a material through which the radiation cannot pass and a transmissive wire made of a material through which radiation can pass. The connection is made by welding the wires together to form a base wire, and the base wire is coiled to form the coil spring.

### BRIEF DESCRIPTION OF THE DRAWINGS

FIG. 1 to FIG. 4 relate to an embodiment of the present invention.

FIG. 1 is a partial side sectional view of the guide wire.

FIG. 2a is a partial side view to illustrate the situation before welding.

FIG. 2b is a partial side view to illustrate the situation after welding and before stretching.

FIG. 2c is a partial side view to illustrate the situation after stretching.

FIG. 3a is an enlarged partial side sectional view to illustrate the situation after welding.

FIG. 3b is an enlarged partial side sectional view to illustrate the situation after stretching.

FIG. 4a is a side view of the curved guide wire.

FIG. 4b is a side view of the curved guide wire in the blood vessel.

FIG. 5 and FIG. 6 show a conventional guide wire.

FIG. 5 is a partial side sectional view of the guide wire.

FIG. 6a is a side view of the curved guide wire.

FIG. 6b is a side view of the curved guide wire in the blood vessel.

FIG. 7 is a diagrammatic view of apparatus for applying a proper welding load in accordance with the invention.

### DETAILED DESCRIPTION

FIG. 1 to FIG. 4 relate to an embodiment of the present invention. A guide wire 101 consists of a flexible main wire 102 and a coil spring 103 in which the top of the main wire 102 is inserted. The coil spring 103 comprises an untransmissive coil part 104 through which radiation cannot pass and a transmissive coil part 105 through which radiation can pass. The untransmissive coil part 104 comprises an untransmissive wire 107 made of a material through which radiation cannot pass. Such material is, for example, a platinum-nickel alloy (platinum 93% by weight: nickel 7% by weight), and the transmissive coil part 105 comprises a transmissive wire 108 made of a material through which radiation can pass. Such material is, for example, an austenite stainless steel (SUS316).

The untransmissive wire 107 of the coil part 104 and the transmissive wire 108 of the coil part 105 are connected together by welding to form a base wire 110 having a diameter of 0.072 mm.

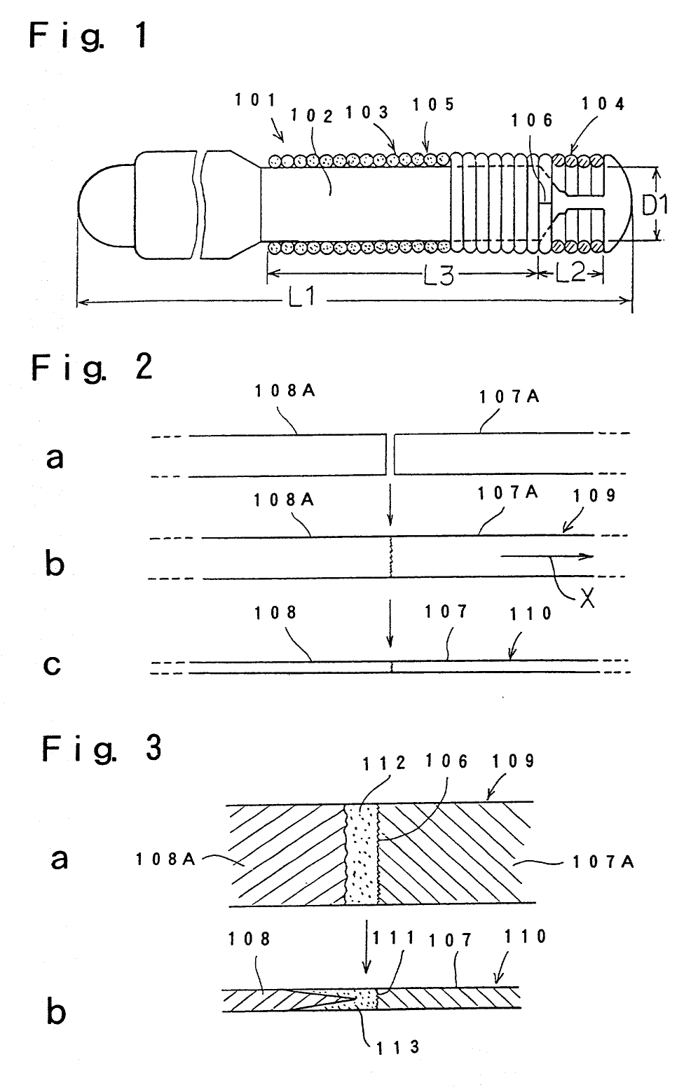

To connect the untransmissive wire 107 and the transmissive wire 108 respectively to form a base wire 110, first, as shown in FIG. 2a, one end of a mother wire 107A of the untransmissive wire 107 having a diameter of 0.3 mm and a proper length and one end of a mother wire 108A of the transmissive wire 108 having a diameter of 0.3 mm and a proper length are set oppositely in a welding apparatus, and resistance welding is effected on the mother wires 107A, 108A to weld the ends of the mother wires 107A, 108A together. A proper welding load is applied to the mother wires 107A, 108A respectively to form a mother base wire 109 as shown in FIG. 2b. Since the welding part of the mother wire 107A and the mother wire 108A has a larger diameter than the original diameter (0.3 mm) of the mother wires 107A, 108A, abrasive finishing is effected on the mother base wire 109 to obtain a uniform diameter.

A proper welding load for purposes of the invention is within the range of 0.3 to 2.0 kg/mm². In one embodiment of the invention, the range is 0.55 to 1.7 kg/mm². In a preferred embodiment of the invention, the range is 0.8 to 1.4 kg/mm².

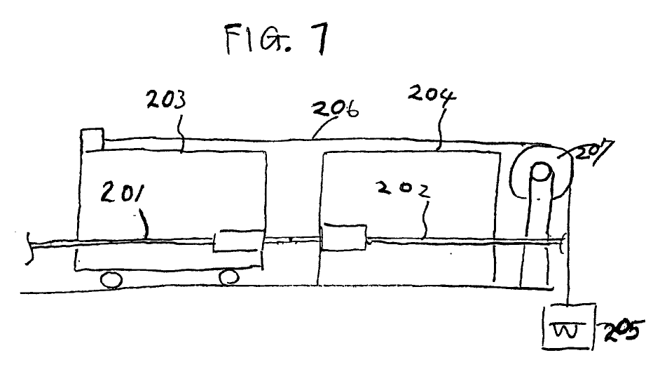

A proper welding load can be applied by the apparatus of FIG. 7. In FIG. 7, one wire 201 is supported on a movable supporting block 203, and the other wire 202 is supported on a fixed supporting block 204. The welding load is applied to the wires 201 and 202 by a weight 205 through a wire 206 and pully 207. The welding load can be adjusted by adjusting value of the weight 205, as those skilled in the art will understand.

The mother base wire 109 having a uniform diameter by the abrasive finishing is stretched by using wire stretching machine as shown by an arrow X in FIG. 2b to obtain the base wire 110 having a diameter of 0.072 mm and then the coil spring 103 is produced from the base wire 110 by the coil-processing.

When the main wire 102 is inserted into the coil spring 103, the coil spring 103 is oriented so that the untransmissive coil part 104 of the coil spring 103 is directed to the top of the main wire 102. In this embodiment, the length L1 of the guide wire 101 is 1780 mm, the inside diameter D1 of the coil spring 103 is 0.193 mm, the length L2 of the untransmissive coil part 104 is 20 mm, and the length L3 of the transmissive coil part 105 is 285 mm respectively, as shown in FIG. 1.

Since the end of the untransmissive mother wire 107A and the opposed end of the transmissive mother wire 108A are connected by welding, platinum atoms and nickel atoms contained in the mother wire 107A diffuse into the mother wire 108A, which contains no platinum atoms and relatively few nickel atoms and is made of a stainless steel, to form a diffusion layer 112 in the connecting part 106 between the mother wire 107A and the mother wire 108A by the thermal energy of the welding process as shown in FIG. 3a. When the mother base wire 109 is stretched, the diffusion layer 112 is stretched at the same time to form a stretched diffusion layer 113 in the connecting part 111 between the resulting untransmissive wire 107 and the resulting transmissive wire 108. Since the mechanical property of the untransmissive wire 107 changes successively to the mechanical property of the transmissive wire 108 in the stretched diffusion layer 113, the stretched diffusion layer 113 acts as a mechanical relaxation zone.

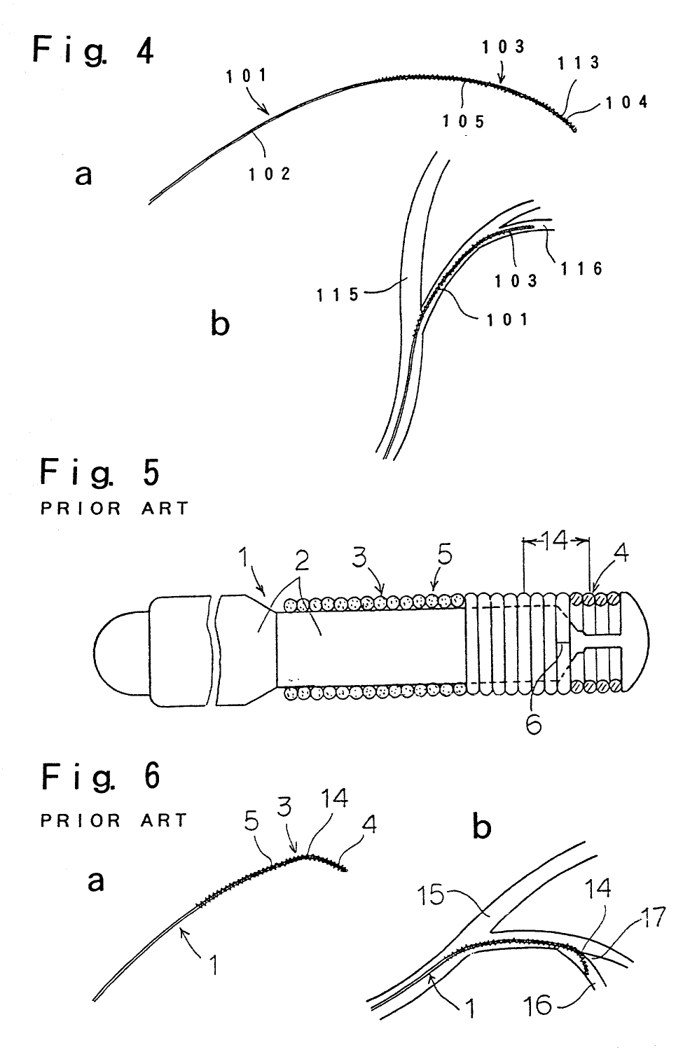

The top of the guide wire 101 inserted in the coil spring 103 curves smoothly because the curvature of the untransmissive coil part 104 and the curvature of the transmissive coil part 105 do not change abruptly. The guide wire 101 therefore has a substantially uniform curvature without forming an angular part as shown in FIG. 4a and is very easy to insert or extract into or from the main blood vessel 115 and the branched blood vessel 116 as shown in FIG. 4b. This prevents damage to the blood vessel 115, 116.

Since the untransmissive wire 107 of the untransmissive coil part 104 and the transmissive wire 108 of the transmissive coil part 105 are strongly connected together through the stretched diffusion layer 113, the guide wire 101 is stable and of high quality without an appreciable risk of breaking in the connecting part 111 between the untransmissive coil part 104 and the transmissive coil part 105.

Since first the mother wire 107A and the mother wire 108A are connected together to form the base wire 110 and then the base wire 110 is coiled in accordance with the present invention, very fine and troublesome work to connect a pair of very small coils together by welding can be avoided the production of the guide wire is therefore rationalized and the production cost is reduced.

Since the coil spring 103 comprises the untransmissive wire 107 made of a platinum-nickel alloy and has a smaller spring back quantity and the transmissive wire 108 is made of an austenite stainless steel having a bigger spring back quantity, the outer diameter of the untransmissive coil part 104 is smaller than the outer diameter of the transmissive coil part 105 wherein there is no stress on the coil spring 103. The top of the guide wire has a bullet shape to be easily inserted or extracted into or from the blood vessel and easily reach the narrow part of the blood vessel.

Since the transmissive wire 108 made of austenite stainless steel has high resistance, the welding can be advantageously performed and the transmissive wire 108 has a good cold hardening property to obtain a coil spring having an excellent performance.

We claim:

1. A guide wire used for medical treatment and comprising a flexible main wire and a coil spring into which the main wire is inserted, the coil spring comprising an untransmissive wire having a relatively small spring back quantity and a transmissive wire having a relatively large spring back quantity, the untransmissive wire being made of a material through which radiation cannot pass and the transmissive wire being made of a material through which radiation can pass, wherein the untransmissive wire and the transmissive wire are connected together by welding with a proper welding load followed by stretching to form a diffusion layer between the untransmissive wire and the transmissive wire and wherein the mechanical property of the untransmissive wire changes successively to the mechanical property of the transmissive wire in the diffusion layer.

2. A guide wire in accordance with claim 1 wherein the untransmissive wire is made of an alloy selected from the group consisting of a platinum alloy, a gold alloy, a tungsten alloy and a lead alloy.

3. A guide wire in accordance with claim 1 wherein the transmissive wire is made of an austenite stainless steel.

4. A guide wire in accordance with claim 1 wherein the main wire has a leading end and the untransmissive coil part is adjacent the leading end.

5. A guide wire according to claim 1 wherein the proper welding load is within the range of 0.3 to 2.0 kg/mm².

6. A guide wire according to claim 1 wherein the proper welding load is within the range of 0.55 to 1.7 kg/mm².

7. A guide wire according to claim 1 wherein the proper welding load is within the range of 0.8 to 1.4 kg/mm².

8. A method of manufacturing a guide wire used for medical treatment, the method comprising:
providing an untransmissive wire made of a material through which radiation cannot pass;
providing a transmissive wire made of a material through which radiation can pass;
connecting the untransmissive wire and the transmissive wire together under a proper welding load;
welding the untransmissive wire and the transmissive wire together; and
stretching the welded wire to form a diffusion layer between the untransmissive wire and the transmissive wire;
whereby the mechanical property of the untransmissive wire changes successively to the mechanical property of the transmissive wire in the diffusion layer; and
coiling the stretched wire to form a coil; and
wrapping the coil around a flexible main wire to form a device for insertion into a blood vessel.
